(19) United States
(12) Patent Application Publication
(10) Pub. No.: US 2013/0324791 A1
(43) Pub. Date: Dec. 5, 2013

(54) METHOD AND DEVICE FOR TREATING PELVIC ORGAN PROLAPSE

(71) Applicants: Magnus Murphy, Calgary (CA); David J. Quinlan, Victoria (CA); Carlos Vonderwalde, Richmond (CA); Roy P. Jackson, Vancouver (CA); Lyndsay Lescisin, Vancouver (CA)

(72) Inventors: Magnus Murphy, Calgary (CA); David J. Quinlan, Victoria (CA); Carlos Vonderwalde, Richmond (CA); Roy P. Jackson, Vancouver (CA); Lyndsay Lescisin, Vancouver (CA)

(21) Appl. No.: 13/989,538

(22) PCT Filed: Nov. 25, 2011
(86) PCT No.: PCT/CA11/01311

§ 371 Date: Aug. 19, 2013

Related U.S. Application Data
(60) Provisional application No. 61/417,262, filed on Nov. 25, 2010.

(51) Int. Cl. A61F 2/00

(52) U.S. Cl. CPC A61F 2/0045
USPC 600/37

(57) ABSTRACT

The present disclosure provides a method of repairing pelvic organ prolapse in a patient, comprising locating a suitable attachment point for fixing a prolapsed vagina to a support tissue in the prespinous area; providing a tissue interface; inserting the interface into the subfascial space of the pelvis through the vaginal epithelium; and positioning the interface and attaching to the attachment point and the rear fascia of the vaginal epithelium. The disclosure further provides a device comprising an elongate hollow element, having a distal end and a proximate end, of a stiffness and length selected to permit insertion of at least a portion of the device from within a subjects vagina through the vaginal epithelium into the subfascial space of the pelvis; and a detachable tissue interface suitable for attaching to the rear fascia of the vaginal epithelium and support tissue within the subfascial space of the pelvis.

### FIELD

This disclosure relates to the treatment of pelvic organ prolapse (POP). In particular, this disclosure relates to a method of treating vaginal prolapse, as well as kits, apparatus, the like.

### BACKGROUND

Pelvic organ prolapse (POP) is a very common condition which occurs in about 50% of all women who have had vaginal childbirth. POP mainly results from damage and the breakdown of support structures within the pelvis. Normally the pelvic organs are kept in place by muscle and connective tissue attachments, for instance ligaments, but in pelvic organ prolapse these structures fail in their functions. The result is the protrusion of vaginal tissue outside the vaginal opening. This protrusion can be secondary to protrusion of the anterior vaginal wall (cystocele), protrusion of the posterior vaginal wall (rectocele or enterocele), or protrusion of the uterus or top of the vaginal wall (uterine or vault prolapse). In some severe cases there can be a complete eversion of the vagina which may protrude completely outside of the body, through the labia.

Pelvic organ prolapse may arise for a variety of reasons such as accidental trauma, surgery such as a hysterectomy, weakening due to age or disease, obesity, frequent constipation, pelvic organ tumours, or the like but is most often linked to the strain and damage caused by vaginal child birth. Older women are more likely to suffer from a prolapse and the condition sometimes run in families.

A range of symptoms are associated with pelvic organ prolapse such as a feeling of pressure from the organs pressing against the vaginal wall, feeling very full in the lower abdomen, urinary incontinence, or inability to empty the bladder completely, pain in the vagina, constipation, incomplete or difficult bowel emptying, pain, and the like.

Current treatment options range from dietary and lifestyle changes and exercises to strengthen the pelvic muscles (Kegels) for mild symptoms; use of a pessary for more severe cases; to surgery for serious cases.

Various kinds of surgery are used for treating pelvic organ prolapse depending on which organ has prolapsed. These types include, for example, vaginal vault prolapse surgery, cystocele surgery, urethrocele surgery, rectocele surgery, enterocele surgery, vaginal obliteration, and hysterectomy.

During vaginal vault surgery, the surgeon usually makes incisions in the walls of the vagina and then tries to reattach the support tissues to their original positions. This can be difficult, can be dangerous to underlying structures, and is quite often unsuccessful. As a result various prosthetic meshes have been developed in an attempt to more strongly compensate for the damage of the native tissue and the inability to recreate the original support.

These are mostly invasive procedures requiring general or spinal anaesthesia and prolonged operative time.

The surgery can be effective but the patient is subjected to considerable pain during the surgery and healing as well as being at risk of complications such as incontinence, urinary retention, pain during sex, infection, bladder or bowel injury, erosion or the formation of a fistula. Furthermore, the prolapse can return after surgery and, on average, 30% of subjects having surgery will require a further surgery within 4 years. In addition, in a three year period in the United States and Canada the use of the mesh has been associated with over 1,000 adverse events prompting the FDA and Health Canada to issue public health notifications regarding the “serious complications” associated with transvaginal implantation of mesh for the treatment of pelvic organ prolapse. These known procedures also require significant surgical expertise in the area of pelvic reconstructive surgery. The surgery and follow-on care during the patient's recovery both tend to be relatively expensive and are particularly burdensome for women due to pain, vaginal discharge or bleeding, and the prolonged time required for bladder and bowel function to return. These operations all require long training periods and extensive learning curves to become competent. As a result, they are usually performed by a small minority of gynecologists and as a result, the needs of the population as a whole is barely met today and will definitely not be met in the future, as the baby boomer generation ages.

Various proposals have been made regarding different treatment options for organ prolapse such as, for example, US 2005/0199249; US 2009/023982; WO 2010/028242; US 2008/0027271; WO 2007/084411; U.S. Pat. No. 3,871,368; WO 2010/017291; WO 2007/025296; US 2007/0068538; WO 2007/097994; WO 2010/051273; WO 2009/038781; and U.S. Pat. No. 7,175,591.

There exists a need for alternative treatment options for pelvic organ prolapses and stress urinary incontinence. Preferred treatment options would remedy the condition in a safe and durable manner and may include one or more of the following characteristics: simple, minimally invasive, reduced chance of infection, reduced incidence of pain, and/or other complications.

### SUMMARY

The present disclosure provides a method of treating a pelvic organ prolapse. The present method may be used to treat an apical prolapse (vault prolapse, uterine prolapse). The present method may be used to treat most cases of anterior prolapse where there is a para-vaginal component.

The present method comprises, in a subject suffering from pelvic organ prolapse,

(a) locating a suitable attachment point for fixing a prolapsed vagina to prespinous support tissue preferably bilaterally (i.e. on both sides of the pelvis);

(b) providing a tissue interface;

(c) inserting the interface into the sub fascial space of the pelvis through the vaginal epithelium; and

(d) positioning the interface and attaching to the attachment point and the rear fascia of the vaginal epithelium.

The present method may provide a relatively durable fixation of the vaginal fascia to the attachment point via a relatively quick and/or minimally procedure.

The present disclosure provides a delivery device suitable for delivering a tissue interface through the vaginal epithelium without major incision or dissection of the vaginal epithelium. The device comprises a shaft and tissue interface, the interface being detachable.

As used herein, “pelvic organ prolapse” refers to the to a cystocele, rectocele, enterocele and/or vaginal vault or uterine prolapse.

As used herein, “tissue interface” refers to a biocompatible substance or device that provides a permanent, semi-permanent, or temporary attachment between the rear fascia of the vaginal epithelium and the attachment point. The attachment should be durable enough to provide relief from the symptoms of prolapse and will preferably result in the formation of an enduring bond between the rear fascia and the attachment point. The present interface will preferably not penetrate into the rear fascia of the vaginal epithelium. The present interface will preferably not penetrate into the tissue of the attachment point.

As used herein, “tissue anchor” refers to a biocompatible device that can provide a permanent or semi-permanent attachment into resilient tissue such as bone or ligament. In the present disclosure the tissue anchor attaches the tissue interface to the attachment point.

As used herein, “attachment point” refers to a position or positions within the subjects body which can provide a place for attaching the vaginal epithelium. For example, the immediate pre-spinous area (origin of the uterosacral ligaments); iliococcygeous muscle tendon, medial to the ischial spine; and/or along the arcus tendineus fascia pelvis (white line of the pelvis where the paravaginal support failed). These are the preferred attachment points since they are some of the anatomically correct and safe areas for attachment. However, in some instances it might be appropriate to attach at other points such as attaching the apex to the sacrospinous ligament.

As used herein, the term “treatment” means any manner in which a pelvic organ prolapse in a subject is eliminated, ameliorated, or otherwise beneficially altered. “Treatment” as used herein is intended to encompass prophylactic treatment of subject at risk of developing a prolapse.

As used herein, the term “subject” is not limited to a specific species or sample type. For example, the term “subject” may refer to a patient, and frequently a human patient. However, this term is not limited to humans and thus encompasses a variety of mammalian species.

As used herein, “a” or “an” means “at least one” or “one or more”.

This summary does not necessarily describe all features of the invention. Other aspects, features and advantages of the invention will be apparent to those of ordinary skill in the art upon review of the following description of specific embodiments of the invention.

### BRIEF DESCRIPTION OF THE DRAWINGS

FIG. 1 shows two possible configurations of a tissue interface.

FIG. 2 shows an option for a proposed delivery system.

FIG. 3 shows a tissue interface mounted on the delivery system.

FIG. 4 shows possible methods of attaching the tissue interface to the delivery system.

FIG. 5 shows perforating needles capable of deploying a securing means for a tissue interface to the rear fascia of a vaginal epithelium.

FIG. 6 shows possible fixation methods for a tissue interface to the rear fascia of a vaginal epithelium.

FIG. 7 shows a configuration of a device that could be used to fix the interface to the ligament.

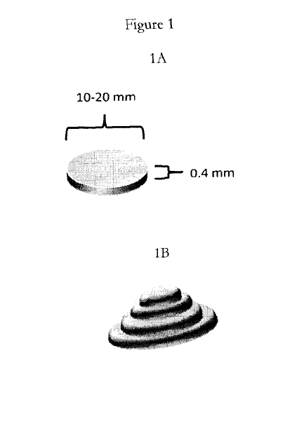

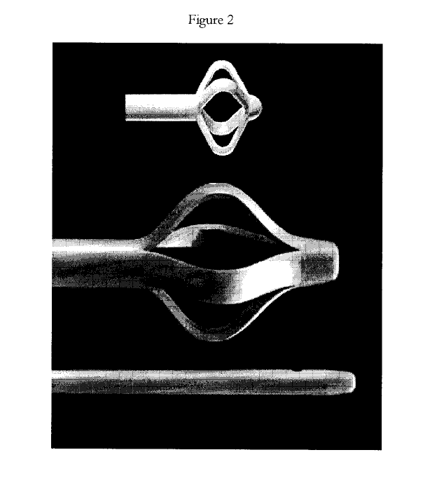

### DETAILED DESCRIPTION

The present disclosure provides a method of treating pelvic organ prolapse. In particular the present disclosure provides, in a subject suffering from or at risk of a pelvic organ prolapse, a method of correcting a pelvic organ prolapse using a tissue interface attached to the rear fascia of the vaginal epithelium and a suitable attachment point.

The present method comprises, in a subject suffering from pelvic organ prolapse, locating a suitable attachment point for fixing the prolapsed vagina to the prespinous area. For example, the vaginal apex may be attached to a suitable position or positions in the prespinous area bilaterally and/or along the pelvic side wall (arcus tendineus). The attachment point or points should be such that, once fixed in place, the vaginal vault is in a relatively normal well-supported position.

The present method may further comprise traversing the vaginal epithelium and inserting a tissue interface through the vagina into the subfascial space of the pelvis. The vaginal epithelium may be punctured in any suitable manner. The interface may be deployed using a device as described herein or through other deployment means.

The present disclosure provides a tissue interface. The interface may be any suitable shape. For example, the interface may be a disc, ring, oblong, square, rectangle, star-shaped, a cross, and the like. The interface may have any suitable three dimensional cross section. For example, the interface may have a conical or concertinaed shape which may provide some shock-absorbing capacity. While not wishing to be bound by theory, it is believed that because the pelvis, in conjunction with all its organs, is in frequent motion (e.g. walking, running, during intercourse etc.) a rigid structure may be damaged more easily by erosion and/or tearing if all the stress is applied to the securing point. However if the structure is built in such a way that will absorb some of this energy it is less likely to cause erosion-damage.

The interface may be any suitable size. For example, the interface may have a diameter of about 0.5 mm or greater, about 1 mm or greater, about 2 mm or greater, about 3 mm or greater, about 4 mm or greater, about 5 mm or greater, about 6 mm or greater, about 7 mm or greater, about 8 mm or greater, about 9 mm or greater, about 10 mm or greater. The interface may have a diameter of about 50 mm or less, about 40 mm or less, about 30 mm or less, about 25 mm or less, about 20 mm or less. The interface may have a thickness of about 0.05 mm or greater, about 0.1 mm or greater, about 0.2 mm or greater, about 0.3 mm or greater, about 0.4 mm or greater, about 0.5 mm or greater. The interface may have a thickness of about 25 mm or less, about 20 mm or less, about 15 mm or less, about 10 mm or less, about 5 mm or less.

The interface may be made of any suitable material. For example, the material may be biological or synthetic. Examples of synthetic materials include PTFE, polyester, Teflon™, Dacron™, polypropylene, and the like. Examples of biological originated materials include pericardium, peritoneum, skin, fascia leather, xenografts derived from but not limited to bovine, equine, porcine species and pericardial, fascia, endothelial, and/or other organs or structures. Autologous or allografts may also be used herein. The tissue interface may be provided using tissue culture techniques to provide auto, allo, or xeno grafts.

The interface may be made from a material that is deployed in the form of a liquid, gel or a suspension which then form a solid or semi-solid once in-situ or in conjunction with such a suspension material. The interface may be made from a tissue adhesive material. The interface may be made from an absorbable material such that, once it has served it function of creating a bond between the rear facia of the vaginal epithelium and the prespinous area, it is absorbed.

Once in the subfascial space of the pelvis the tissue, the interface forms a bond between the rear facia of the vaginal epithelium and the prespinous area. The bond may be achieved in any suitable manner. For example, the interface may be adhesive to the tissue, sutured in position, attached with a barbed suture, stapled in position, any mechanical means, or combinations thereof. Various means have been disclosed in the art that may be suitable for this purpose including barbed elements, corkscrews, umbrella-types, screws, and the like. The tissue anchors disclosed in US 2005/0199249; US 2009/023982 may also be appropriate. Some specific, non-limiting examples include using a bone anchor such as Autosuture ProTack™, Aptus™ endostapling system, or the like.

It is preferred in the present method that the tissue interface is deployed at or around the vaginal apex. However, it may be deployed at any suitable location. Furthermore, the vagina may be fixed in position at one or more other positions either with additional interfaces or with other means. For example, tissue adhesives, such as fribrinogen based adhesive, may be introduced to one or more positions providing a secondary support means attaching the repositioned vagina.

Non-limiting examples of tissue adhesives include Tisseel™ (fribrinogen, thrombin, aprotinin) and BioGlue™ (purified bovine serum albumin and glutaraldehyde).

An embodiment of the present method comprises returning the displaced vaginal vault to an appropriate position by, for example, manual manipulation. Once the vagina is repositioned a suitable attachment point is located in support tissue such as the sacrospinous ligament or prespinous area. Other support tissue may also be appropriate such as the levator muscle, the ileococcygeus fascia, the white line, the faschia over the obturator internus muscle, and combinations thereof. After the attachment point or points are located an incision or puncture is made in the corresponding vaginal epithelium. A tissue interface is inserted through this incision into the subfascial space of the pelvis. For example, the interface may be inserted with a suitable delivery device. The interface is then fixed in position through, for example, sutures. Various suturing systems are known such as, for example, Neatstich™, Anulex™, the Ct Closure system, and the like and detached from the delivery device. Once the interface is fixed in placed an inflatable pessary may be positioned in the vagina and inflated.

The present disclosure provides a delivery device or stylet. The delivery device comprises a shaft and a detachable tissue interface as described herein. The device may be any size and shape suitable for delivering the interface through the vaginal epithelium to the subfascial space of the pelvis. The device has a distal and a proximal end. Preferably the device has a diameter at the distal end of about 2 cm or less, about 1.5 cm or less, about 1 cm or less, about 50 mm or less, about 40 mm or less, about 30 mm or less, about 20 mm or less. Preferably the shaft is at least about 10 cm in length, at least about 20 cm in length, at least about 25 cm in length, at least about 30 cm in length. The shaft may be provided with a plurality of visually or tactilely perceivable markings that enable the user to ascertain the depth of penetration into the subject's body and its orientation. The proximal end may comprise a graspable handle for ease of use. The distal end preferably comprises the tissue interface. The distal end may be pointed in order to facilitate its movement through the vaginal epithelium.

The interface may be held on the delivery device by slits, spikes, sutures, any other suitable releasable mechanism, or combinations thereof. The delivery device preferably comprises a means for triggering the release of the interface once it is in the correct position. For example, the device may comprise retractable spikes which hold the interface to the device until retracted. The trigger for the release mechanism may be at the distal or, more preferably, the proximal end of the device.

The device may comprise a means for delivering tissue glue to the proximity of the interface. For example, the device may comprise a dedicated channel through which the glue may be delivered.

The device may comprise a means for attaching a syringe which may, for example, contain a tissue glue and/or an antibiotic solution.

The device may be manufactured out of any suitable material. For example, the device may be manufactured out of a disposable material. Such materials are well known in the art. Preferably the material is a sterilizable, resilient but bendable material such as stainless steel, extruded plastic with a stainless steel core for rigidity and shape-ability.

The present device may comprise an elongate hollow element, having a distal end and a proximal end, of a stiffness and length selected to permit insertion of at least a portion of the device from within a patient's correctly repositioned vagina through a wall of the vagina and to a selected depth into the subfascial space of the pelvis proximate to the selected support tissue; a tissue interface suitable for attaching to the rear fascia or the vaginal epithelium and the support tissue.

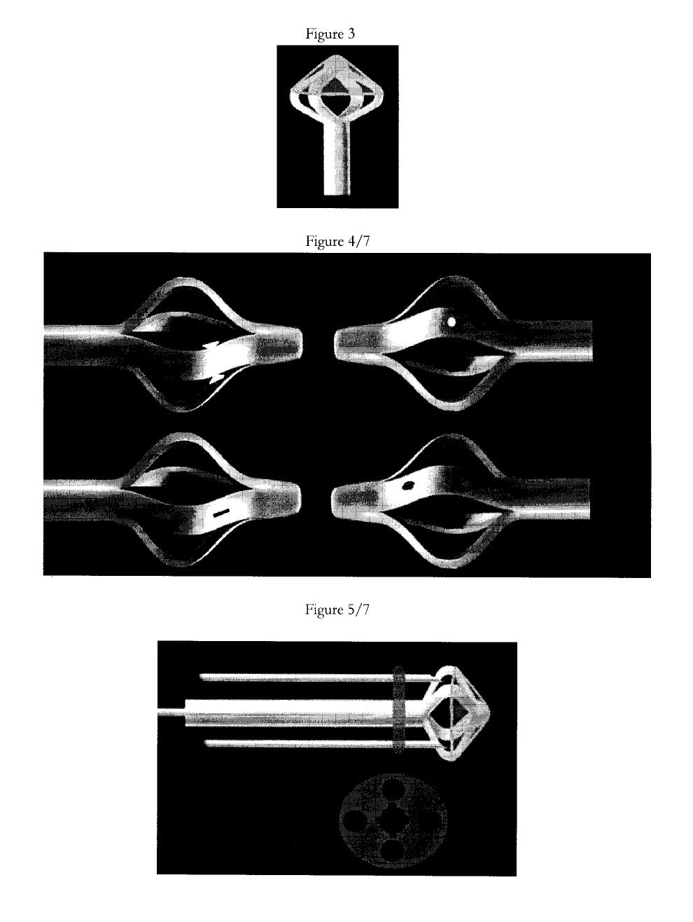

The device may be similar to a Mallecot catheter with a modified inner mandrel that will stretch or retract the catheter to insert and later deploy the interface. The interface can be mounted on the outside of the mechanism so it can be easily detached from the delivery system and left in place without compromising the integrity of the interface. The delivery system may utilise an “umbrella” type mechanism. The mechanism may have two or more, three or more, four or more arms (FIG. 3, 4). The interface may be secured to the inner wall of the vagina at the fornix level by a guided delivery system added to the delivery catheter that will direct the suturing mechanism in between the “umbrella” arms, it will most likely consist of sharp needles to perforate and deliver the suture/anchor.

In an embodiment, the present device is employed in the following manner:

1. Identify the vaginal attachment point and insert the stylet to perforate the vaginal tissue in the selected area, advance the delivery device through the vagina to the prolapsed tissue;

2. Advance the stylet at least partially through the incision and into the subfascial space of the pelvis with the “closed umbrella” configuration with a tissue interface pre-attached at the distal end to maintain a low profile and ease of insertion;

3. Deploy the “open umbrella” configuration;

4. Fix the tissue interface to the sub-epithelial space by deploying perforating needles, once fixed then detach the tissue interface from the delivery device;

5. Attach a pre-filled syringe and deliver surgical glue in to the sub-epithelial space;

6. Withdraw the stylet;

7. Insert an anchoring device to fix the interface to the prespinous selected area, deploy the anchor and, if needed, inject glue between the interface and the attachment point

8. Suture, if necessary, the puncture site with a preferably bio-absorbable suture;

9. Repeat, if necessary, at other locations; and

10. If necessary, place a temporary inflatable pessary in the vagina and inflate.

In an embodiment, the present disclosure comprises:

1. Making a small incision or puncture to drive the delivery catheter from the vaginal potential space to the subfascial space of the pelvis, this can be done at the fornix level. The delivery catheter comprises a tissue interface patch;

2. Advancing a delivery catheter with a pericardial interface attached through the incision/puncture, at this stage the delivery catheter is stretched and the patch presents a low profile (e.g. closed umbrella);

3. Once in the subfascial space of the pelvis the catheter is deployed (e.g. open umbrella) to expand the patch to a circular shape;

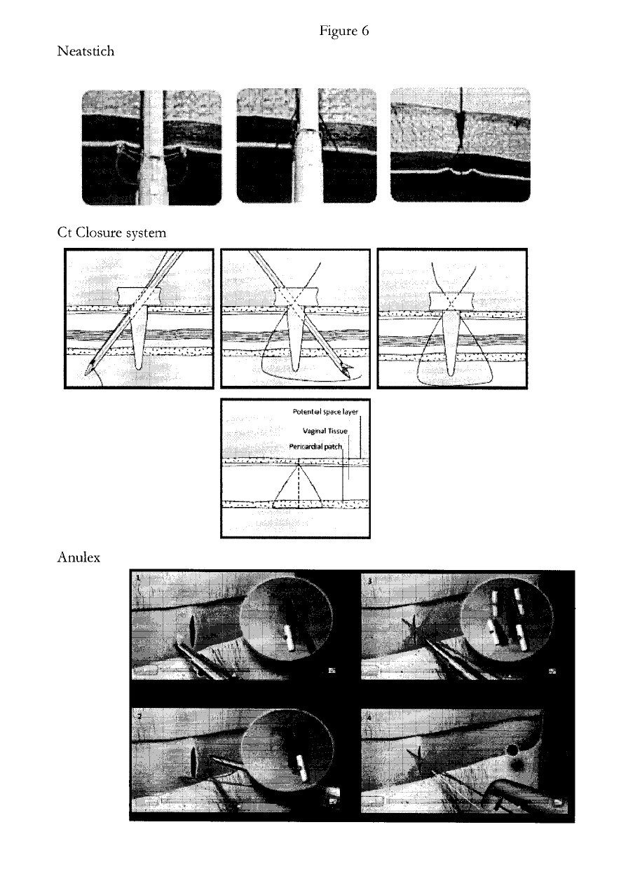

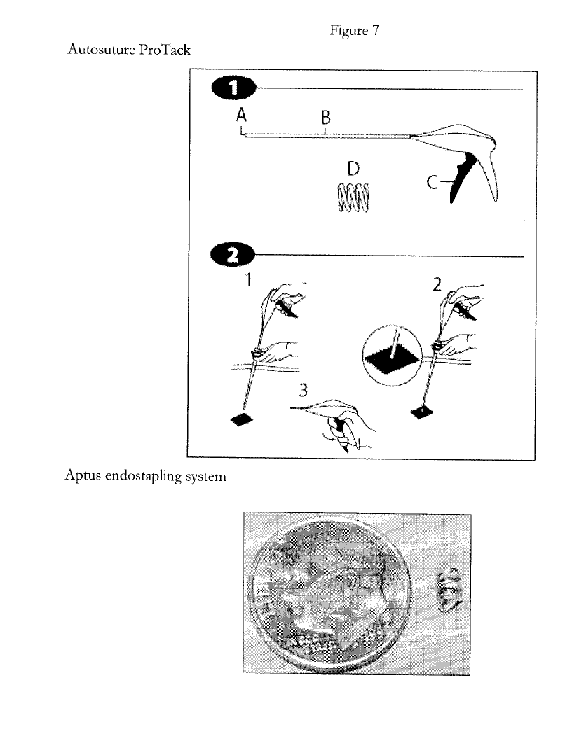

4. The patch is fixed to the inner surface of the vaginal tissue by using one of the suturing mechanism described herein (e.g. FIG. 6);

5. The delivery catheter withdrawn;

6. An endostapler is advanced through the incision/puncture and approximate the vagina by pushing the patch towards the bone. The stapler is then fired;

7. A syringe/needle loaded with biological glue is advanced through the incision/puncture to perforate the patch and the glue applied to the bone/patch junction point, the needle is then withdrawn;

8. The incision/puncture is then closed, if necessary, with regular stitching;

9. Repeat, if necessary, at other locations; and

10. If necessary, place a temporary inflatable pessary in the vagina and inflate.

The present disclosure provides a kit for treating a pelvic organ prolapse, the kit comprising the present delivery device suitable for traversing the vaginal epithelium and at least one tissue interface. The kit may comprise two or more interfaces of different sizes and/or shapes enabling the kit to be used to address a variety of prolapses. The kit will preferably be in a sterile, single-use format and may comprise instructions for treating a pelvic organ prolapse in accordance with the present disclosure.

Based on the extent and type of prolapse the present tissue interface or interfaces may be placed in any of suitable support structures to facilitate a durable repair of the prolapse. The surgeon will determine how many interfaces and/or tissue glue to deploy in which selected support tissues.

It is contemplated that any embodiment discussed in this specification can be implemented or combined with respect to any other embodiment, method, composition or aspect of the invention, and vice versa.

Unless defined otherwise, all technical and scientific terms used herein have the same meaning as is commonly understood by one of ordinary skill in the art to which this invention belongs. Unless otherwise specified, all patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference. Citation of references herein is not to be construed nor considered as an admission that such references are prior art to the present invention.

Use of examples in the specification, including examples of terms, is for illustrative purposes only and is not intended to limit the scope and meaning of the embodiments of the invention herein. Numeric ranges are inclusive of the numbers defining the range. In the specification, the word “comprising” is used as an open-ended term, substantially equivalent to the phrase “including, but not limited to,” and the word “comprises” has a corresponding meaning.

The invention includes all embodiments, modifications and variations substantially as hereinbefore described and with reference to the examples and figures. It will be apparent to persons skilled in the art that a number of variations and modifications can be made without departing from the scope of the invention as defined in the claims. Examples of such modifications include the substitution of known equivalents for any aspect of the invention in order to achieve the same result in substantially the same way.

1. A method for addressing a pelvic organ prolapse in a subject, comprising the steps of:
a. locating a suitable attachment point for fixing a prolapsed vagina to a support tissue in the prespinous area;
b. providing a tissue interface;
c. inserting the interface into the subfascial space of the pelvis through the vaginal epithelium; and
d. attaching the interface to the attachment point and the rear fascia of the vaginal epithelium.

2. A method of addressing a pelvic organ prolapse in a subject, comprising the steps of:
a. manually repositioning the subject's vagina to correct the prolapse;
b. locating a suitable attachment point for the vagina within a support tissue in the subfascial space of the pelvis;
c. penetrating the vaginal epithelium;
d. inserting a tissue interface through the vaginal epithelium;
e. securing the interface to the rear facia of the vaginal epithelium; and
f. securing the interface to the attachment point.

3. The method of claim 2 wherein the penetration of the vaginal epithelium is an incision or puncture.

4. The method of claim 3 wherein the incision or puncture is repaired following the insertion of the tissue interface.

5. A device comprising an elongate hollow element, having a distal end and a proximate end, of a stiffness and length selected to permit insertion of at least a portion of the device from within a subject's vagina through the vaginal epithelium into the subfascial space of the pelvis; and a detachable tissue interface suitable for attaching to the rear fascia or the vaginal epithelium and support tissue within the subfascial space of the pelvis.

6. A kit comprising a device according to claim 5 and instructions for its use.

7. The device of claim 5 where the tissue interface is of biological origin.

8. The device of claim 5 wherein the tissue interface is an autologous graft.

9. The device of claim 5 wherein the tissue interface is a xenograft.

10. The device of claim 5 where the tissue interface is cultured in vitro.

11. The device of claim 5 where the tissue interface is biostable.

12. The device of claim 5 where the tissue interface is biodegradable.

13. The device of claim 5 wherein the tissue interface has a diameter of from about 0.5 mm to about 50 mm and a thickness of from about 0.05 mm to about 25 mm.

14. The device of claim 5 wherein the tissue interface is the shape of a square, circle, ring, cone, rectangular pyramid, or the like.

15. The device of claim 5 wherein the device has a diameter at the distal end of about 2 cm or less, and wherein the device is at least about 10 cm in length.

16. A tissue interface adapted for insertion into the subfascial space of the pelvis, attachment to the rear facia of the vaginal epithelium and an attachment point within the support tissue in the subfascial space of the pelvis, wherein the interface has a diameter of from about 0.5 mm to about 50 mm and a thickness of from about 0.05 mm to about 25 mm.

17. The interface of claim 16 wherein the tissue interface is the shape of a square, circle, ring, cone, rectangular pyramid, or the like.

18. Use of a tissue interface according to claim 16 for the correction of a pelvic organ prolapse.

19. The use of claim 18 wherein the use comprises:
a. locating a suitable attachment point for fixing a prolapsed vagina to a support tissue in the prespinous area;
b. inserting the tissue interface into the subfascial space of the pelvis through the vaginal epithelium; and
c. attaching the tissue interface to the attachment point and the rear fascia of the vaginal epithelium.

20. The use of claim 18 wherein the use comprises:
a. manually repositioning the subject's vagina to correct the prolapse;
b. locating a suitable attachment point for the vagina within a support tissue in the subfascial space of the pelvis;
c. penetrating the vaginal epithelium;
d. inserting the tissue interface through the vaginal epithelium;
e. securing the interface to the rear facia of the vaginal epithelium; and
f. securing the interface to the attachment point.
